(19) United States
(12) Patent Application Publication
(10) Pub. No.: US 2014/0178970 A1
(43) Pub. Date: Jun. 26, 2014

(54) GENERATION OF MATERIALS WITH ENHANCED HYDROGEN CONTENT FROM ANAEROBIC MICROBIAL CONSORTIA INCLUDING DESULFUROMONAS OR CLOSTRIDIA

(71) Applicant: Luca Technologies, Inc., Golden, CO (US)

(72) Inventor: Gary Vanzin, Arvada, CO (US)

(73) Assignee: Luca Technologies, Inc., Golden, CO (US)

(21) Appl. No.: 14/049,034

(22) Filed: Oct. 8, 2013

Related U.S. Application Data
(63) Continuation of application No. 13/189,030, filed on Jul. 22, 2011, now abandoned.
Application No. 13/189,030 is a continuation of application No. 11/971,075, filed on Jan. 8, 2008, now abandoned.
Application No. 11/971,075 is a continuation-in-part of application No. 11/099,881, filed on Apr. 5, 2005, now abandoned.
Application No. 11/099,881 is a continuation-in-part of application No. 11/099,880, filed on Apr. 5, 2005, now abandoned.

(51) Int. Cl. C12N 1/20

(52) U.S. Cl. CPC C12N 1/20
USPC 435/252.1

(57) ABSTRACT

An isolated microbial consortia is described. The consortia may include a first-bite microbial consortium that converts a starting hydrocarbon that is a complex hydrocarbon into two or more first-bite metabolic products. The consortia may also include a downstream microbial consortium that converts a starting hydrocarbon metabolic product into a downstream metabolic product. The downstream metabolic product has a greater mol. % hydrogen than the starting hydrocarbon. The first-bite microbial consortium or the downstream microbial consortium includes one or more species of Desulfuromonas.

### CROSS REFERENCES TO RELATED APPLICATIONS

This application is a continuation of application Ser. No. 13/189,030, filed Jul. 22, 2011 and entitled “Generation of Materials With Enhanced Hydrogen Content From Anaerobic Microbial Consortia Including Desulfuromonas Or Clostridia,” which is a continuation of application Ser. No. 11/971,075, filed Jan. 8, 2008, and entitled “Generation of Materials With Enhanced Hydrogen Content From Anaerobic Microbial Consortia,” which is a continuation-in-part of prior application Ser. No. 11/099,881, filed Apr. 5, 2005, and entitled “Generation Of Materials With Enhanced Hydrogen Content From Anaerobic Microbial Consortia.” This application is also a continuation-in-part of prior application Ser. No. 11/099,880, filed Apr. 5, 2005, and entitled “Generation Of Materials With Enhanced Hydrogen Content From Anaerobic Microbial Consortia Including Thermotoga.” The entire contents of both applications are herein incorporated by reference for all purposes.

### FIELD OF THE INVENTION

The present invention relates to biogenic enhancement of the mole percentage of hydrogen in hydrocarbon molecules and enhancements in biogenic hydrogen and methane production in geologic formations. Specifically, the invention relates to isolated microbial consortia that can include archaea, bacteria, and/or other microorganisms, which are capable of transforming carbonaceous materials in the formations into molecular hydrogen, and/or hydrocarbons having a larger mole percentage of hydrogen than the starting materials.

### BACKGROUND OF THE INVENTION

Increasing world energy demand is creating unprecedented challenges for recovering energy resources, and mitigating the environmental impact of using those resources. Some have argued that the worldwide production rates for oil and domestic natural gas will peak within a decade or less. Once this peak is reached, primary recovery of oil and domestic natural gas will start to decline, as the most easily recoverable energy stocks start to dry up. Historically, old oil fields and coal mines are abandoned once the easily recoverable materials are extracted. These abandoned reservoirs, however, still contain significant amounts of carbonaceous material. The Powder River Basin in northeastern Wyoming, for example, is still estimated to contain approximately 1,300 billion short tons of coal. Just 1% of the Basin's remaining coal converted to natural gas could supply the current annual natural gas needs of the United States (i.e., about 23 trillion cubic feet) for the next four years. Several more abandoned coal and oil reservoirs of this magnitude are present in the United States.

As worldwide energy prices continue to rise, it may become economically viable to extract additional oil and coal from these formations with conventional drilling and mining techniques. However, a point will be reached where more energy must be used to recover the resources than is gained by the recovery. At that point, traditional recovery mechanisms will become uneconomical, regardless of the price of energy. Thus, new recovery techniques are needed that can extract resources from these formations with significantly lower expenditures of energy.

Conventional recovery techniques also extract the carbonaceous materials in their native state (e.g., crude oil, coal), and the combustion products of these materials may include a number of pollutants, including sulfur compounds (SOₓ), nitrogen compounds (NOₓ), and carbon dioxide (CO₂). Concern about the environmental impact of burning these native carbonaceous materials has led to national and international initiatives to develop less polluting energy sources. One approach is to generate more energy with natural gas (i.e., methane), which has low levels of sulfur and nitrogen, and generates less carbon dioxide per unit energy than larger hydrocarbons.

Another approach that is receiving considerable government and private sector support is the development of hydrogen engines and fuel cells for vehicle propulsion and electricity generation. The combustion of molecular hydrogen (H₂) into water presents a more benign environmental alternative to burning gasoline, oil or coal. Hydrogen, however, is more accurately characterized as an energy carrier than a fuel source. Very little molecular hydrogen exists in nature, and other energy sources are needed to make the hydrogen. The role of hydrogen is to carry the energy from another energy source to the site where it can be released by chemical reaction (e.g., combustion) to do useful work. A power and transportation infrastructure based on hydrogen will require adequate supplies of energy and/or feedstock materials to make the hydrogen. One well known method of making hydrogen is the steam reforming of methane, where methane (CH₄) and steam (H₂O) are converted into carbon monoxide (CO) and hydrogen (H₂). Thus, one way to realize a hydrogen economy will be economically converting large quantities of methane to hydrogen and recover it.

The above discussion and citation of documents herein is not intended as an admission that any is pertinent prior art. All statements as to the date or representation as to the contents of documents is based on the information available to the applicant and does not constitute any admission as to the correctness of the dates or contents of the documents.

### BRIEF SUMMARY OF THE INVENTION

The present invention relates to microorganisms that participate in the degradation of large or complex hydrocarbons found in naturally occurring sources, such as those present in underground formations. The microorganisms are useful for the recovery of energy contained within large or complex hydrocarbons, many of which are associated with other materials that hinder extraction of the hydrocarbons from the formations, by converting the hydrocarbons to smaller molecules that can be more readily recovered or extracted.

The invention is based in part on energy recovery by conversion of large or complex hydrocarbons to smaller hydrocarbons, optionally with release thereof from materials that hinder extraction of large or complex hydrocarbons. The route is based on biogenic conversion of carbonaceous materials in underground formations, which conversion has received relatively little commercial attention. Large potential sources of energy, locked up in carbonaceous materials such as (but not limited to) coal, residual oil, etc., may be more readily recovered by conversion of the hydrocarbons in the carbonaceous materials, as well as the carbonaceous material itself, into methane and other light hydrocarbons. In biogenic conversion, consortia of microorganisms treat the carbonaceous materials as a source of raw materials for conversion into smaller, lighter metabolites including alcohols, organic acids, aromatic compounds, molecular hydrogen, and/or methane as non-limiting examples. Conversion by microorganisms includes their reformation or utilization of starting materials to form products by metabolism, including catabolism and/or anabolism by microorganisms of a consortium.

Given that in in situ in sub-surface formations, the concentrations of free oxygen (i.e., O₂) often falls below the level that can sustain aerobic metabolism in microorganisms (or strict aerobic microorganisms), it is possible that consortia of anaerobic microorganisms (including obligate and/or facultative anaerobic microorganisms or microaerophiles) predominate. Unfortunately, most anaerobic microorganisms cannot survive in the oxygen rich atmosphere above ground, and are difficult to study in conventional laboratories. For this reason and others, anaerobic consortia of microorganisms that can metabolize carbonaceous materials are poorly understood. The invention is based upon the identification and isolation of consortia members that participate in the biogenic conversion of carbonaceous material, as well as the hydrocarbons therein, into molecules with a higher molar percentage (mol. %) of hydrogen atoms than in the carbonaceous material or hydrocarbons therein. Non-limiting examples of molecules with a high mol. % of hydrogen atoms include molecular hydrogen (H₂) and methane (CH₄). The isolated consortia of the invention may also be modified to have enhanced abilities (e.g., an increased metabolic rate as a non-limiting example) to convert starting materials to hydrocarbons with a higher mol. % of hydrogen atoms.

In a first aspect, the invention provides microorganisms that have been isolated from the environment in which they are naturally found, such as, but not limited to, those isolated from a geologic formation comprising other organisms and/or other chemical compounds found in the formation. In some embodiments, the microorganisms may be isolated by reducing or removing one or more environmental compounds found with the microorganisms. For example, if the native microorganism environment is the water present in the formation, then reducing the concentration of a hydrocarbon (e.g., methane, oil, etc.) in extracted formation water produces isolated consortia of the microorganisms in the water. Similarly, reducing the concentration of one or more other molecules from a sample or preparation of such water results in an isolate of microorganisms therein as an embodiment of the invention. Non-limiting examples of such molecules include carbon dioxide, one or more amines, one or more nitrates, one or more nitrites, one or more alcohols, one or more organic acids, one or more sulfates, one or more sulfites, hydrogen, hydrogen sulfide (H₂S), one or more halogen ions (e.g., Cl⁻ and/or Br⁻ ions), and/or one or more metal ions (e.g., ions of alkali metals, alkali earth metals, transition metals, etc.) may also produce isolated consortia of the microorganisms from the formation water. Isolated consortia may be produced as the formation water flows through a purification and/or extraction system that removes the compound(s) before being pumped back into the same, or a different geological formation. Isolated consortia may also be produced by extracting the native formation water to a storage container, and removing the compound(s) from the stored water.

The isolated microorganisms are in the form of a consortium, comprising a plurality of two or more different species of microorganisms. In some embodiments, a consortium of the invention contains two or more different microorganisms that are metabolically related, such as where the microorganisms have a symbiotic relationship with each other. The invention includes consortia wherein two or more of the species of microorganisms present therein are related by syntrophy such that one microorganism is a syntroph of one or more others. Such consortia are advantageous where individual syntroph microorganisms cannot be separately cultured or propagated (in the absence of the related syntroph(s)).

Embodiments of the invention include isolated microbial consortia for biogenically increasing the hydrogen content of a product derived from a starting hydrocarbon that includes complex hydrocarbons that make up a carbonaceous material like coal or oil. The consortia includes a first-bite microbial consortium that converts the starting hydrocarbon into two or more first-bite metabolic products. The consortia also includes a downstream microbial consortium that converts a starting hydrocarbon metabolic product into a downstream metabolic product. The downstream metabolic product has a greater mol. % hydrogen than the starting hydrocarbon. The first-bite microbial consortium or the downstream microbial consortium include one or more species of Desulfuromonas.

Embodiments of the invention also include isolated microbial consortia for biogenic methane production. The consortia may include a first microbial consortium that converts a starting hydrocarbon into one or more intermediate compounds. The consortia may also include a second microbial consortium that converts at least one type of the intermediate compounds into CO₂ and H₂. The consortia may further include a third microbial consortium that converts the CO₂ and H₂ into methane and water. At least one of the first, second and third microbial consortiums comprises at least one species of Desulfuromonas.

Embodiments of the invention may still further include isolated microbial consortia for biogenic methane production that use an acetate metabolism step. The consortia may include a first microbial consortium that converts a starting hydrocarbon into one or more intermediate compounds, and a second microbial consortium that converts at least one type of the intermediate compounds into acetate. The consortia may additionally include a third microbial consortium that converts the acetate into methane and water. At least one of the first, second and third microbial consortiums comprises at least one species of Desulfuromonas.

In another aspect, the invention provides a consortium derived from a consortium isolated from a naturally occurring source. Non-limiting examples of such a derivative consortium include those that have a different composition of microorganisms due to selection by culture conditions as well as those that have one or more non-naturally occurring microorganisms due to mutation that occurred during culture or maintenance of the consortium.

An additional aspect of the invention provides methods of making a microbial consortia that biogenically increases hydrogen and/or methane content of products derived from a carbonaceous source material. Thus a consortium of microorganisms that does not have the capability of increasing hydrogen and/or methane content may be modified by the invention to have that capability. Alternatively, a consortium that has the capability may be modified to increase that capability. The invention provides a method of preparing a modified (or augmented) consortium comprising the addition of at least one species of the genus Desulfuromonas to an unmodified (or unaugmented) first consortium. The addition may be by the addition of a second consortium, containing a species of Desulfuromonas, to said first consortium. The method may be preceded by the isolation of the species of Desulfuromonas or isolation of a microbial consortium that contains the species.

Where a second consortium is used to prepare a modified (or augmented) consortium, the second consortium may include microorganisms capable of converting or metabolizing the carbonaceous source material into a first set of one or more intermediate hydrocarbons. The second consortium may also include a microbial consortium capable of converting the intermediate hydrocarbons into a second set of intermediate hydrocarbons. The hydrocarbons of the second set of intermediate hydrocarbons may or may not have a higher mol. % of hydrogen atoms than the first set of intermediate hydrocarbons. In addition to the addition of a second consortium, a modified consortium may further include a third microbial consortium that converts the second set of intermediate hydrocarbons into smaller hydrocarbons and other metabolites such as water and/or carbon dioxide. In some embodiments, the smaller hydrocarbons have a greater mol. % of hydrogen atoms than the starting carbonaceous source material.

In a further aspect, methods for the use of a microbial consortium of the invention are provided. In some embodiments, a consortium of the invention is introduced into a geological formation to result in the production of molecular hydrogen and/or methane by their metabolic activities. The introduction maybe accompanied by, preceded by, or followed by, introduction of one or more agents to into the formation to result in conditions, in all or part of the formation, conducive to the growth of microorganisms the consortium. In other embodiments, a consortium of the invention may be used in a method of stimulating a microbial consortia endogenous to a geological formation to increases hydrogen and/or methane production from a carbonaceous source material in the formation. In additional embodiments, the method includes the introduction of one or more species of Desulfuromonas microorganisms, alone or in a consortium comprising them, to the in situ environment of a group of native microorganisms that are metabolizing the carbonaceous source material. The method may also include changing an environmental condition in at least part of the formation to enhance the growth of the one or more Desulfuromonas species and/or additional consortia of microorganisms introduced into the formation to increase the population of the microbial consortia that biogenically increases hydrogen and/or methane production from the carbonaceous source material in the formation. The changed environmental condition, or other condition for the microorganisms, may include temperature, pH, oxidation potential (Eh), microorganism nutrient concentrations, salinity, and metal ion concentrations, among other environmental conditions.

In yet another aspect of the invention, embodiments of consortia and methods as described herein may include an identified microorganism other than Desulfuromonas. They may, for example, include species from the genera Thermotoga, Gelria, Clostridia, Moorella, Thermacetogenium, Pseudomonas, Methanobacter or other species of microorganism with the same capabilities as the microorganisms and consortia described herein.

For example, embodiments of the invention may include isolated microbial consortia for biogenically increasing the hydrogen content of a product derived from a starting hydrocarbon that includes complex hydrocarbons that make up a carbonaceous material like coal or oil. The consortia includes a first-bite microbial consortium that converts the starting hydrocarbon into two or more first-bite metabolic products. The consortia also includes a downstream microbial consortium that converts a starting hydrocarbon metabolic product into a downstream metabolic product. The downstream metabolic product has a greater mol. % hydrogen than the starting hydrocarbon. The first-bite microbial consortium or the downstream microbial consortium include one or more species of Fusibacter and/or Acetobacterium.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

FIG. 1 shows a simplified schematic of the biogenic conversion of carbonaceous materials to methane according to embodiments of the invention;

FIG. 2 shows a flowchart with method steps for making and measuring the characteristics of a consortia according to embodiments of the invention;

FIG. 3 is plot of the methanogensis rate (tmols of methane/gram of coal/day) as a function of the percentage of Desulfuromonas in a microorganism consortium; and

FIG. 4 is a plot of the methanogensis rate (tmols of methane/gram of coal/day) as a function of the percentage of Fusibacter in a microorganism consortium.

### DETAILED DESCRIPTION OF THE INVENTION

Anaerobic consortia are described that can convert starting hydrocarbons in native carbonaceous materials into hydrocarbons having a greater mol. % of hydrogen atoms, such as methane. In some embodiments, such consortia contain microorganisms that do not require molecular oxygen as a terminal electron acceptor in their combined metabolism, but rather can perform methanogenesis as the final electron accepting step to produce methane. In their native state, carbonaceous materials such as coals and oils contain complex, polymeric hydrocarbons with multiple saturated and unsaturated carbon-carbon, carbon-nitrogen, carbon-sulfur, and carbon-oxygen bonds. The hydrocarbons are also large, which as used herein refers to hydrocarbons of more than 20 carbon atoms and/or 400 g/mol molecular weight. Moreover, and as used herein, “hydrocarbon” refers to molecules containing only carbon and hydrogen atoms, optionally containing one or more nitrogen, sulfur, and oxygen atoms. The invention provides microorganisms and consortia comprising them to convert the complex and/or large hydrocarbons into smaller molecules, including smaller hydrocarbons with less than 20 carbon atoms and/or 400 g/mol molecular weight.

When the microorganisms of a consortium as described herein convert these complex and/or large starting hydrocarbons into smaller hydrocarbons, the ratio of C—C to C—H bonds is typically reduced, resulting in higher mol. % of hydrogen atoms for the product molecules because of an increase in the number of hydrogen atoms relative to the number of non-hydrogen atoms in a product molecule. For example, acetic acid has the chemical formula CH₃COOH, representing 2 carbon atoms, 2 oxygen atoms, and 4 hydrogen atoms, to give a total of 8 atoms. Since 4 of the 8 atoms are hydrogen, the mol. % of hydrogen atoms in acetic acid is: (4 Hydrogen Atoms)/(8 Total Atoms)=0.5, or 50%, by mol. (or on a molar basis). Methane has the chemical formula CH₄, representing 1 carbon atom and 4 hydrogen atoms, making a total of 5 atoms. The mol. % of hydrogen atoms in methane is (4 Hydrogen Atoms)/(5 Total Atoms)=0.8, or 80%, by mol. Thus, the conversion of acetic acid to methane increases the mol. % of hydrogen atoms from 50% to 80%. In the case of molecular hydrogen, the mol. % of hydrogen atoms is 100%. The invention includes microorganisms, as well as consortia and methods of using them, wherein the net increase in the mol. % of hydrogen atoms, starting from a complex and/or larger hydrocarbon to a final smaller hydrocarbon, is from less than about 66% to 80 or 100%, from about 66% to 80 or 100%, or from about 70% to 80 or 100%.

In some embodiments, each step of a microorganism or consortium's metabolic pathway increases the mol. % of hydrogen atoms of the resultant metabolite. For example, in a three-step metabolic pathway where: (1) a portion of the starting hydrocarbons in the native carbonaceous material are metabolized into a phenol; (2) the phenol is metabolized into acetic acid; and (3) the acetic acid is metabolized into methane, the mol. % of hydrogen atoms increases at each step. In other embodiments, intermediate steps in the metabolic pathway may decrease the mol. % of hydrogen atoms. For example, another three-step metabolic pathway may include the metabolic steps of: (1) converting native carbonaceous material to acetic acid; (2) converting the acetic acid to hydrogen (H₂) and carbon dioxide (CO₂); and (3) converting the H₂ and CO₂ into methane and water. For this metabolic pathway, the mol. % of hydrogen atoms goes from 100% for H₂, to 80% for methane, which represents a decrease in the mol. % hydrogen between steps (2) and (3). However, there is still an increase in the mol. % hydrogen between the starting carbonaceous materials and the final metabolic product (i.e., methane).

The biogenically produced hydrocarbons produce fewer pollutants than the native carbonaceous materials, including less sulfur and nitrogen oxides, and fewer volatile organic compounds (VOCs) caused by incomplete combustion of polymeric hydrocarbons. Moreover, the lower concentration of carbon relative to hydrogen in these hydrocarbons means less carbon dioxide is produced upon combustion for an equivalent amount of energy, reducing the rate at which this greenhouse gas is added to the atmosphere.

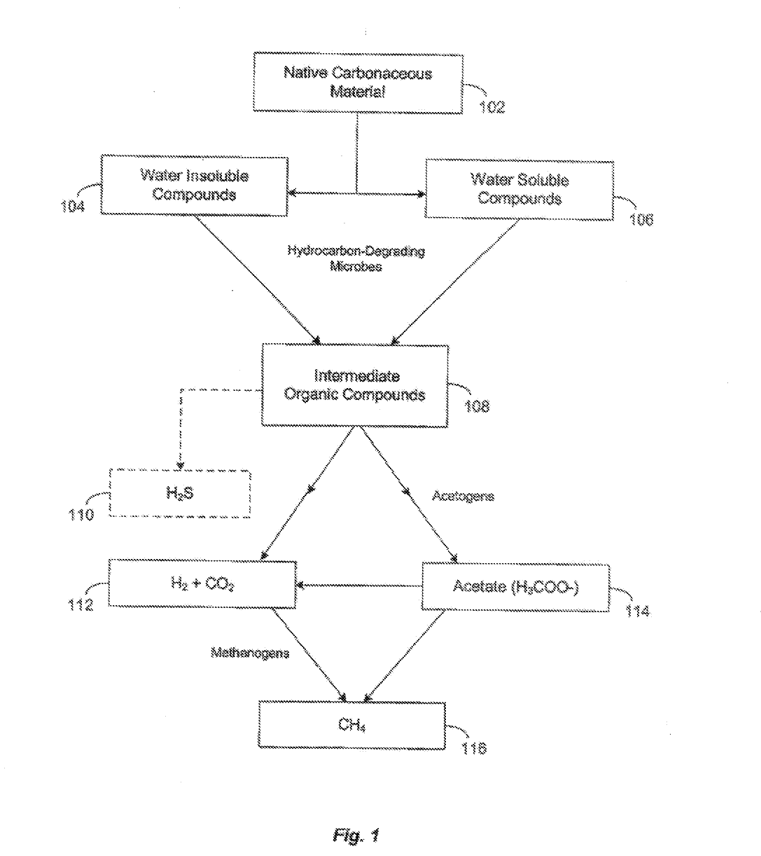

Referring now to FIG. 1, a simplified schematic of the biogenic conversion of starting hydrocarbons in carbonaceous materials to methane is shown. Native carbonaceous material 102 such as oil, coal, coke, kerogen, anthracite, coal tar, bitumen, lignite, peat, carbonaceous shale, and sediments rich in organic matter, among others, may include polymers 104 that are insoluble in the surrounding formation water, and other polymers 106, such as partially water soluble polyaromatics, that are present in the formation water as well as in solid substrate.

Hydrocarbon-degrading microorganisms metabolize the solid polymers 104 and/or the aqueous polymers 106 into intermediate organic compounds 108, such as alkanes, alkenes, alkynes, aromatic compounds, alcohols, organic acids, and amines, among others. For example, native carbonaceous materials like oil, which are predominantly composed of saturated and unsaturated alkyl hydrocarbons, the organic compounds may include straight-chained or branched, alkanes, alkenes, and alkynes. The metabolites may also include substituted and unsubstituted hydrocarbons, such as ethers, aldehydes, ketones, alcohols, organic acids, amines, thiols, sulfides, and disulfides, among others. For native carbonaceous materials like coal, which have large, complex arrays of highly unsaturated, benzene-like rings linked together, the depolymerization products may include substituted and unsubstituted, mono- and poly-aromatic hydrocarbons, including benzenes, naphthalenes, anthracenes, phenanthrenes, coronenes, etc.; substituted aromatics such as alkyl aromatics (e.g., toluene, xylene, styrene) aromatic alcohols (e.g., phenol), aromatic amines (e.g., aniline), aromatic aldehydes (e.g., benzaldehyde), aromatic acids (e.g., benzoic acid). etc.; and substituted and unsubstituted heterocyclic aromatic groups, such as pyridines, pyrroles, imidazoles, furans, thiophenes, quinolines, indoles, etc.

Additional examples of depolymerization products may include acetylene, 1,1,1-Trichloro-2,2-bis-(4-chlorophenyl)ethane, acrylonitrile, 2-Aminobenzoate, 1,3-Dichloropropene, Dichloromethane, Dimethyl sulfoxide, Carbazole, Benzoate, p-Xylene, p-Cymene, Carbon tetrachloride, Fluorene, Adamantanone, 3-Chloroacrylic Acid, 2-Chloro-N-isopropylacetanilide, 1,4-Dichlorobenzene, Parathion, Toluene, Octane, Nitrobenzene, 4-Chlorobiphenyl, Dibenzothiophene, Orcinol, Xylene, Ethylbenzene, Mandelate, Styrene, Trichloroethylene, Toluene-4-sulfonate, m-Xylene, Atrazine, Naphthalenesulfonates, 2,4-Dichlorobenzoate, Chlorobenzene, 2-Aminobenzoic Acid, 4-Chlorobiphenyl, Ethylbenzene, Naphthalene, Chlorobenzene, 1-Aminocyclopropane-l-carboxylate, Biphenyl, Caprolactam, Phenanthrene, 2,4,6-Trinitrotoluene, m-Cresol, Thiocyanate, Phenylmercuric chloride, n-Octane, Dodecyl Sulfate, Bromoxynil, and Dibenzothiophene, among other products.

Of course each of the above described compounds may be produced in free form in the general environment outside microorganisms of a consortium or in secluded form in or in between particular microorganisms of a consortium. This is particularly appropriate in the context of some syntrophically related microorganisms, which may pass one or more of the above compounds between each other rather than diffusion into the general environment beyond the microorganisms.

The intermediate organic compounds 108 may then be further metabolized into a number of metabolites, including hydrogen sulfide (H₂S) 110, hydrogen (H₂) and carbon dioxide (CO₂) 112, and acetic acid (i.e., acetate) 114. The quantity and types of metabolites produced depend on the make-up of the microorganism or consortium used to convert the intermediate organic compounds. For example, consortia dominated by thiophillic microorganisms favor the production of hydrogen sulfide 110, while consortia dominated by acetogens and/or methanogens favor the production of acetate 114 and methane (CH₄) 116, respectively.

Methane 116 may be produced from intermediate organic compounds 108 by a number of metabolic pathways. In some pathways, microorganisms may break down the organic compounds 108 directly into hydrogen and carbon dioxide 112. From this point, methanogens in the consortia may convert the hydrogen and carbon dioxide 112 into methane. In another pathway, the organic compounds are first converted by acetogens into acetate 114 and/or formate (HCO—). Microorganisms in the consortia may then transform or convert the acetate directly into methane 116 and CO₂, or first convert the acetate into hydrogen and CO₂ 112, which methanogens then convert to methane 116 and water.

For complex consortia made up of 10 or more, 20 or more, 30 or more different species of microorganisms, it will be appreciated that the conversion of one metabolite to another may involve a plurality of microorganisms using a plurality of metabolic pathways to metabolize a plurality of intermediate compounds.

Consortia described herein may be made up of one or more consortia (or subpopulations) of microorganisms, where each consortium (or subpopulation) may be identified by the role that the consortium plays in the overall conversion of starting carbonaceous materials to an end product. Each consortium (or subpopulation) includes a plurality of microorganisms that may belong to the same or different genus or belong to the same or different species. When a consortium (or subpopulation) includes a plurality of different species, individual species may work independently or in concert to carry out the role of the consortium. The term microorganism as used here includes bacteria, archaea, fungi, yeasts, molds, and other classifications of microorganisms. Some microorganisms can have characteristics from more than one classification (such as bacteria and fungi), and the term microorganism used here also encompasses these hybrid classifications of microorganisms.

Because sub-surface formation environments typically contain less free atmospheric oxygen (e.g., O₂) than found in tropospheric air, consortia are described as anaerobic consortia. These anaerobic consortia are consortia that can live and grow in an atmosphere having less free oxygen than tropospheric air (e.g., less than about 18% free oxygen by mol.). In some instances, anaerobic consortia operate in a low oxygen atmosphere, where the O₂ concentration is less than about 10% by mol., or less than about 5% by mol., or less than about 2% by mol., or less than about 0.5% by mol. The formation water may also contain less dissolved oxygen than what is typically measured for surface water (e.g., about 16 mg/L of dissolved oxygen). For example, the formation water may contain about 1 mg/L or less of dissolved oxygen.

The microorganisms that make up the consortia may include obligate anaerobes that cannot survive in an atmosphere with molecular oxygen concentrations that approach those found in tropospheric air (e.g., 18% to 21%, by mol. in dry air) or those for which oxygen is toxic. Consortia may also include facultative aerobes and anaerobes that can adapt to both aerobic and anaerobic conditions. A facultative anaerobe is one which can grow in the presence or absence of oxygen, but grow better in the presence of oxygen. A consortium can also include one or more microaerophiles that are viable under reduced oxygen conditions, even if they prefer or require some oxygen. Some microaerophiles proliferate under conditions of increased carbon dioxide of about 10% mol or more (or above about 375 ppm). Microaerophiles include at least some species of Thermotoga and Giardia.

In some embodiments, the ratio of aerobes to anaerobes in consortia may change over time. For example, consortia may start in an environment like oxygenated water before being introduced into a sub-surface anaerobic formation environment. Such consortia start out with higher percentages of aerobic microorganisms and/or facultative anaerobes (such as an aerobic consortium of Bacillus and/or Geobacillus bacteria that metabolize the carbonaceous substrate of the formation into fermentation products) that use the molecular oxygen in fermentation processes to metabolize carbonaceous materials in the formation. As the molecular oxygen concentration decreases, growth of the aerobes is slowed as anaerobic microorganisms or consortia metabolize the aerobic fermentation products into organic compounds with higher mol. % of hydrogen atoms.

Consortia embodiments may be described by dividing the consortia into three or more consortia defined by the function they play in the conversion of starting hydrocarbons in native carbonaceous materials (like coal and oil) into end hydrocarbons like methane. The first microbial consortium (or subpopulation) of the consortia includes one or more microorganisms that break down the starting hydrocarbons into one or more intermediate organic compounds. For example, when the carbonaceous material is bituminous coal, one or more microorganisms of the first consortium may split an alkyl group, or aromatic hydrocarbon from the polymeric hydrocarbon substrate. This process may be referred to as the metabolizing of the carbonaceous material, whereby the complex macromolecular compounds found in the carbonaceous material are decomposed into lower molecular weight hydrocarbon residues.

The second microbial consortium (or subpopulation) includes one or more microorganisms that metabolize or otherwise transform the intermediate organic compounds into other intermediate organic compounds, including compounds with oxidized, or more highly oxidized, carbons (e.g., alcohol, aldehyde, ketone, organic acid, carbon dioxide, etc.). These second stage intermediate organics are typically smaller, and may have higher mol. % of hydrogen atoms, than the starting organic compounds, with one or more carbons being split off as an oxidized carbon compound. “Oxidized carbon” refers to the state of oxidation about a carbon atom wherein an order of increasingly oxidized carbon atoms is from —C—H (carbon bonded to hydrogen); to —C—OH (carbon bonded to a hydroxyl group, such as an alcohol as a non-limiting example); —C═O (carbon double-bonded to oxygen); —COOH (carbon as part of a carboxyl group); and CO₂ (carbon double-bonded to two oxygen atoms) which is the most oxidized form of carbon. As a carbon atom is more oxidized, the total energy associated with the bonds about that atom decreases. This is consistent with the general tendency that as microorganisms extract energy from carbon containing molecules, they remove hydrogen atoms and introduce oxygen atoms. As used herein, “oxidized carbon” does not include any carbon atom that is only bonded to hydrogen and/or one or more carbon atoms.

Because carbon dioxide is generally considered to contain no obtainable energy, the present invention is based in part on the advantageous use of microorganisms to convert the carbon atom in carbon dioxide into a higher energy state, such as in methane. This may be considered a reversal of the oxidation process that produced carbon dioxide by members of a consortium of the invention.

The third microbial consortium (or subpopulation) includes one or more microorganisms that metabolize the final intermediate organic compounds into at least one smaller hydrocarbon (having a larger mol. % hydrogen than the intermediate hydrocarbon) and water. For example, the final intermediate compound may be formate (HCO—) that is metabolized by members of the third consortium into methane and water. This is another example of a reversal of the oxidation process that led to formate. Consortia according to these embodiments include at least one consortium of microorganisms that do not form methane by the pathway of reducing carbon dioxide to methane. This consortium may co-exist in the consortia with other consortia that produce methane by reducing carbon dioxide to produce methane.

In other embodiments, consortia may include one or more consortium (or subpopulations) having different functions than those described above. For example, consortia may include a first consortium that breaks down the starting hydrocarbons in the carbonaceous material into one or more intermediate organic compounds, as described above. The second consortium, however, metabolizes the intermediate organics into carbon dioxide and molecular hydrogen (H₂). A third consortium, which includes one or more methanogens, may convert CO₂ and H₂ into methane and water.

A consortia may include intraconsortium and interconsortium syntrophic interactions. For example, members of the second and third consortia above may form a syntrophic acetate oxidation pathway, where acetate is converted to methane at an enhanced metabolic rate. Microorganisms in the second consortium convert acetic acid and/or acetate (H₃CCOO⁻) into carbon dioxide and hydrogen, which may be rapidly metabolized by methanogens in the third consortium into methane and water. Removal of second consortium metabolites (e.g., hydrogen, carbon dioxide) by members of the third consortium prevents these metabolites from building up to a point where they can reduce metabolism and growth in the second consortium. In turn, the second consortium provides a steady supply of starting materials, or nutrients, to members of the third consortium. This syntrophic interaction between the consortia results in the metabolic pathway that converts acetate into methane and water being favored by the consortia. Syntrophic interactions may also be formed between microorganism populations at other points in a metabolic process, and may be established between members within a consortium (i.e., an intraconsortium interaction), as well as between members of different consortia (i.e., and interconsortium interaction). For example, a syntrophic interaction may exist between acetogens, which form the acetate, and the microorganisms that oxidize the acetate into carbon dioxide and hydrogen. In metabolic processes with multiple steps, several syntrophic interactions may occur down the pathway from reactants to products.

Thus as used herein, syntrophy refers to symbiotic cooperation between two metabolically different types of microorganisms (partners) wherein they rely upon each other for degradation of a certain substrate. This often occurs through transfer of one or more metabolic intermediate(s) between the partners. For efficient cooperation, the number and volume of the metabolic intermediate(s) has to be kept low. In one non-limiting example pertinent to the present invention, syntrophs include those organisms which oxidize fermentation products from methanogens, such as propionate and butyrate, that are not utilized by the methanogens. These organisms require low concentrations of molecular hydrogen to ferment substrates to carbon dioxide, so are symbiotic with methanogens, which help maintain low molecular hydrogen levels.

Native anaerobic consortia have been collected from a variety of sub-surface formations, and studied in a controlled, low-oxygen environment to classify the functions of each consortium that make up the consortia, as well as the microorganisms that make up each consortium. Rates of biogenic hydrocarbon production have also been compared between consortia to identify microorganisms, and combinations of consortia that are particularly effective at converting carbonaceous materials into other hydrocarbons that have higher mol. % hydrogen. Isolation of these microorganisms as consortia has led to the embodiments of the present invention, which include an isolated microbial consortia comprising a first microbial consortium capable of converting large and/or complex starting hydrocarbons into a product comprising one or more first intermediate hydrocarbons; a second microbial consortium, comprising one or more species of Desulfuromonas, capable of converting one or more of the first intermediate hydrocarbons into a product comprising one or more second intermediate hydrocarbons and one or more molecules comprising oxidized carbon; and a third microbial consortium capable of converting one or more of the second intermediate hydrocarbons into a product comprising one or more smaller hydrocarbons and water, wherein the smaller hydrocarbons have a greater mol. % hydrogen than the large and/or complex hydrocarbons.

In these embodiments, the large and/or complex starting hydrocarbons may be those of a carbonaceous source material, such as coal, oil, kerogen, peat, lignite, oil shale, tar sands, bitumen, and tar as non-limiting examples. Moreover, the product comprising one or more first intermediate hydrocarbons may contain a molecule selected from an organic acid, an alcohol, an amine, a straight or branched hydrocarbon, and an aromatic hydrocarbon. The product comprising one or more second intermediate hydrocarbons may contains a molecule selected from formate, acetate, and benzoate. In some particular embodiments, the one or more smaller hydrocarbons comprises methane. In other embodiments, the molecules comprising oxidized carbon comprises CO and/or CO₂.

In many embodiments of the invention, a consortium comprises bacteria and/or archaea (archaebacteria). The first, second, or third microbial consortium of the invention may comprise or consist of one or more obligate anaerobic microorganism or facultative anaerobic microorganism or microaerophile as described herein. Alternatively, the first, second, and third microbial consortium may each comprise or consist of one or more obligate anaerobic microorganism or facultative anaerobic microorganism or microaerophile.

In some embodiments, the first microbial consortium comprises microorganisms of the genera Desulfuromonas, Pseudomonas, Bacillus, Geobacillus, and/or Clostridia, while the second microbial consortium comprises microorganisms of the genera Desulfuromonas, Thermotoga, Pseudomonas, Gelria and/or Moorella. Alternatively, the second consortium may comprise Thermacetogenium, such as Thermacetogenium phaeum. The third microbial consortium may comprise microorganisms of the genus Desulfuromonas and/or Methanobacter, such as, but not limited to, Methanobacter thermoautotrophicus and/or Methanobacter wolfeii. In another example, the third microbial consortium may comprise microorganisms of the genera Methanosarcina, Methanocorpusculum, Methanobrevibacter, Methanothermobacter, Methanolobus, Methanohalophilus, Methanococcoides, Methanosalsus, Methanosphaera, and/or Methanomethylovorans, among others.

Embodiments of the consortia may also include microorganisms from the genera Granulicatella, Acinetobacter, Fervidobacterium, Anaerobaculum, Ralstonia, Sulfurospirullum, Acidovorax, Rikenella, Thermoanaeromonas, Desulfovibrio, Dechloromonas, Acetogenium, Ferribacter, and Thiobacillus, among other microorganisms.

In yet additional embodiments, an isolated microbial consortia for biogenically producing methane from a starting hydrocarbon is provided. This consortia comprises a first microbial consortium to convert the starting hydrocarbon into a product containing one or more intermediate hydrocarbon compounds; a second microbial consortium to convert the intermediate carbon compounds into a product comprising carbon dioxide and molecular hydrogen; and a third microbial consortium to convert the carbon dioxide and molecular hydrogen into methane and water. The first microbial consortium comprises a first group of microorganisms capable of converting the starting hydrocarbon into a product comprising intermediate organic compounds, and a second group of microorganisms capable of converting the intermediate organic compounds into a product comprising smaller organic compounds. At least one of the first, second and third microbial consortiums may include at least one species of Desulfuromonas. For example, the first consortium may include a Desulfuromonas microorganism, the second consortium may include a Desulfuromonas microorganism, and/or the third consortium may include a Desulfuromonas microorganism.

In some embodiments, the intermediate organic compounds comprise aromatic compounds. In other embodiments, the product comprising smaller organic compounds includes a molecule selected from the group consisting of formate, acetate, benzoate, an alcohol, and an organic acid.

In such consortia, the starting hydrocarbon may be that present in crude oil or coal. Non-limiting examples also include those where the starting hydrocarbon is present in a subsurface geological formation, such as that of an oil formation, a natural gas formation, a coal formation, a bitumen formation, a tar sands formation, a lignite formation, a peat formation, a carbonaceous shale formation, and a formation comprising sediments rich in organic matter.

Other isolated microbial consortia for anaerobic production of methane from a larger hydrocarbon include those comprising a first microbial consortium to convert the starting hydrocarbon to form a product comprising smaller hydrocarbons; and a second microbial consortium to convert at least a portion of the smaller hydrocarbons to form a product comprising acetate; and a third microbial consortium to convert said acetate to form methane and water. The third microbial consortium may comprise a first group of microorganisms that convert acetate into carbon dioxide and free hydrogen, and a second group of microorganisms that convert the carbon dioxide and free hydrogen into methane and water. At least one of the first, second and third microbial consortiums may include at least one species of Desulfuromonas. For example, the first consortium may include a Desulfuromonas microorganism, the second consortium may include a Desulfuromonas microorganism, and/or the third consortium may include a Desulfuromonas microorganism.

Microorganisms of the invention identified as being involved in the initial conversion of the carbonaceous material may include aerobes such as Bacillus and Geobacillus bacteria, and anaerobes like Clostridia, among other microorganisms.

The metabolic products, which may also be called anaerobic fermentation products, may be further metabolized into hydrocarbons having a greater mol. % of hydrogen atoms. The microorganisms involve here may include one or more microorganisms from the first consortium and/or other microorganisms to make a second consortium, which metabolize the first metabolic products into additional hydrocarbons and oxidized carbon (e.g., alcohols, organic acids, carbon monoxide, carbon dioxide, etc.). Microorganisms that may be associated with a consortium defined by this metabolic stage may include Desulfuromonas, Pseudomonas, Thermotoga, Gelria (e.g., Gelria glutamica), Clostridia (e.g., Clostridia fervidus), and/or Moorella (e.g., Moorella glycerini, Moorella mulderi) microorganisms.

In addition to Desulfuromonas, Thermotoga species are identified in a number of consortia that are efficient at producing methane from carbonaceous substrate. Specific Thermotoga species identified include Thermotoga hypogea, Thermotoga lettingae, Thermotoga subterranean, Thermotoga elfii, Thermotoga martima, Thermotoga neapolitana, Thermotoga thernarum, and Thermotoga petrophila, among others. Without being bound by theory, and offered to improve understanding of the invention, Thermotoga microorganisms are believed to play a role in the anaerobic oxidation of hydrocarbons to alcohols, organic acids (e.g., acetic acid), and carbon dioxide. For example, a Thermotoga hypogea microorganism in the context of the invention may metabolize a substrate depolymerization product into acetic acid, carbon dioxide, and other organic alcohols and/or acid. Downstream microorganisms may then metabolize the acetic acid into hydrogen (H₂) and carbon dioxide, which is then assimilated into methane and water by another consortium of microorganisms (e.g., methanogens).

Downstream microorganisms that can metabolize the acetic acid include Thermacetogenium microorganisms, such as Thermacetogenium phaeum, which metabolizes the acetic acid into carbon dioxide and hydrogen (H₂). While not wishing to be bound by a particular theory of metabolic action, it is believed that the higher rates of methane production measured for consortia having Thermotoga microorganisms may be attributed to syntrophic interactions between the Thermotoga and downstream microorganisms like Thermacetogenium phaeum, which metabolize acetic acid. The syntrophic interaction may be caused by the Thermotoga and Thermacetogenium microorganisms having similar metabolic responses to environmental characteristics. For example, the microorganisms may have similar metabolic responses to changes in temperature, pH, Eh, nutrient concentrations, etc., that can syntrophically amplify an overall change in the metabolic activity of consortia.

The carbon dioxide and hydrogen may be metabolized into methane and water by a downstream consortium that includes one or more methanogens. The methanogens may include methanogenic archaea such as Methanobacteriales, Methanomicrobacteria, Methanopyrales, and Methanococcales. Methanogenic microorganisms identified in methane producing consortia include Methanobacter thermoautotrophicus, and Methanobacter wolfeii, among others. Here again, while not wishing to be bound by a particular theory of metabolic action, it is believed that a syntrophic interaction may occur between the upstream Thermacetogenium and the downstream Methanobacter to syntrophically enhance the overall metabolic activity of the consortia. The Methanobacter remove hydrogen and carbon dioxide produced by the Thermacetogenium, which prevents a buildup of these materials that could hinder the Thermacetogenium from making additional CO₂ and H₂.

Embodiments of the consortia may include methanogens that metabolize starting materials other that acetate, or carbon dioxide and hydrogen, into methane. For example, the consortia may include methanogens that metabolize alcohols (e.g., methanol), amines (e.g., methylamines), thiols (e.g., methanethiol), and/or sulfides (e.g., dimethyl sulfide) into methane. These may include methanogens from the genera Methanosarcina (e.g., Methanosarcina barkeri, Methanosarcina thermophile, Methanosarcina siciliae, Methanosarcina acidovorans, Methanosarcina mazeii, Methanosarcina frisius); Methanolobus (e.g., Methanolobus bombavensis, Methanolobus tindarius, Methanolobus vulcani, Methanolobus taylorii, Methanolobus oregonensis); Methanohalophilus (e.g., Methanohalophilus mahii, Methanohalophilus euhalobius); Methanococcoides (e.g., Methanococcoides methylutens, Methanococcoides burtonii); and/or Methanosalsus (e.g., Methanosalsus zhilinaeae). They may also be methanogens from the genus Methanosphaera (e.g., Methanosphaera stadtmanae and Methanosphaera cuniculi, which are shown to metabolize methanol to methane). They may further be methanogens from the genus Methanomethylovorans (e.g., Methanomethylovorans hollandica, which is shown to metabolize methanol, dimethyl sulfide, methanethiol, monomethylamine, dimethylamine, and trimethylamine into methane).

In addition, one or more of the consortiums may include microorganisms selected from Desulfuromonadales bacterium JN18_A94_J, Desulfuromonadales bacterium Tc37, Desulfuromonas acetexigens, Desulfuromonas acetoxidans, Desulfuromonas acetoxidans DSM 684, Desulfuromonas alkaliphilus, Desulfuromonas chloroethenica, Desulfuromonas michiganensis, Desulfuromonas palmitatis, Desulfuromonas sp. CD-1, Desulfuromonas sp. FD-1, Desulfuromonas sp. SDB-1, Desulfuromonas sp. SDB-2, Desulfuromonas thiophila, Desulfuromusa bakii, Desulfuromusa kysingii, Desulfuromusa sp. Fe30-7C-S, Desulfuromusa sp. S1, Desulfuromusa succinoxidans, Geoalkalibacter ferrihydriticus, Geobacter argillaceus, Geobacter bemidjiensis, Geobacter bemidjiensis Bem, Geobacter bremensis, Geobacter chapelleii, Geobacter grbiciae, Geobacter hephaestius, Geobacter humireducens, Geobacter hydrogenophilus, Geobacter lovleyi, Geobacter lovleyi SZ, Geobacter metallireducens, Geobacter metallireducens GS-15, Geobacter pelophilus, Geobacter pickeringii, Geobacter psychrophilus, Geobacter sp. CLFeRB, Geobacter sp. ENN1, Geobacter sp. FRC-32, Geobacter sp. M18, Geobacter sp. M21, Geobacter sp. Ply1, Geobacter sp. Ply4, Geobacter sp. TMJ1, Geobacter sp. VES-1, Geobacter sulfurreducens, Geobacter sulfurreducens PCA, Geobacter uraniumreducens, Geobacter uraniumreducens Rf4, Geobacteraceae bacterium JN18_V95_J, Geopsychrobacter electrodiphilus, Geothermobacter ehrlichii, Geothermobacter sp. Fe30-MC-S, Malonomonas rubra, Pelobacter acetylenicus, Pelobacter acidigallici, Pelobacter carbinolicus, Pelobacter carbinolicus DSM 2380, Pelobacter masseliensis, Pelobacter propionicus, Pelobacter propionicus DSM 2379, Pelobacter sp. A3b3, Pelobacter venetianus, and Trichlorobacter thiogenes.

One or more of the consortiums may include a Desulfomicrobium bacteria such as Desulfomicrobium apsheronum, Desulfomicrobium baculatum, Desulfomicrobium escambiense, Desulfomicrobium hypogeium, Desulfomicrobium macestii, Desulfomicrobium norvegicum, Desulfomicrobium orale, Desulfomicrobium sp. 63, Desulfomicrobium sp. ADR21, Desulfomicrobium sp. ADR26, Desulfomicrobium sp. ADR28, Desulfomicrobium sp. ARI902/01, Desulfomicrobium sp. ‘Bendigo B, Desulfomicrobium sp. BL, Desulfomicrobium sp. Bsl6, Desulfomicrobium sp. C4, Desulfomicrobium sp. ‘Clear 59m’, Desulfomicrobium sp. CME2, Desulfomicrobium sp. ‘Delta+’, Desulfomicrobium sp. DsvB, Desulfomicrobium sp. La1.1, Desulfomicrobium sp. MSL65, Desulfomicrobium sp. MSL92, Desulfomicrobium sp. MSL93, Desulfomicrobium sp. MSL94, Desulfomicrobium sp. MSL95, Desulfomicrobium sp. MSL97, Desulfomicrobium sp. MSL98, Desulfomicrobium sp. oral clone BP1-74, Desulfomicrobium sp. P004A, Desulfomicrobium sp. SA2, Desulfomicrobium sp. ‘Scale 10m’; Desulfomicrobium sp. ‘Scale 7m’, Desulfomicrobium sp. ‘Scale 9m’, Desulfomicrobium sp. ‘Scale sediment’, Desulfomicrobium sp. STP10, Desulfomicrobium sp. STP16, and/or Desulfomicrobium thermophilum.

One or more of the consortiums may include a microorganism selected from Desulfacinum subterraneum, Desulfacinum sp. M40/2 CIV-2.3, Desulfacinum hydrothermale, Desulfacinum infernum, Desulfatimicrobium mahresensis, Desulfobacca acetoxidans, Desulfoglaeba sp. Lake, Desulfoglaeba alkanexedens, Desulfomonile limimaris, Desulfomonile tiedjei, Desulforhabdus sp. DDT, Desulforhabdus sp. BKA11, Desulforhabdus amnigena, Desulfovirga adipica, Smithella propionica, Syntrophobacter fumaroxidans MPOB, Syntrophobacter sulfatireducens, Syntrophobacter sp. ECP-C3, Syntrophobacter pfennigii, Syntrophobacter fumaroxidans, Syntrophobacter sp. DSM 10017, Syntrophobacter sp., Syntrophobacter wolinii, Syntrophus aciditrophicus, Syntrophus aciditrophicus SB, Syntrophus sp., Syntrophus gentianae, Syntrophus buswellii, Thermodesulforhabdus sp. NS-tSRB-1, Thermodesulforhabdus n. sp. M40/2 CIV-3.2, and Thermodesulforhabdus norvegica.

One or more of the consortiums may include a microorganism selected from Bilophila wadsworthia, Desulfohalobiaceae bacterium Benz, Desulfohalobium retbaense, Desulfohalobium utahense, Desulfomicrobium apsheronum, Desulfomicrobium baculatum, Desulfomicrobium escambiense, Desulfomicrobium hypogeium, Desulfomicrobium macestii, Desulfomicrobium norvegicum, Desulfomicrobium orale, Desulfomicrobium sp., Desulfomicrobium sp. 63, Desulfomicrobium sp. ADR21, Desulfomicrobium sp. ADR26, Desulfomicrobium sp. ADR28, Desulfomicrobium sp. AR1902/01, Desulfomicrobium sp. Bendigo B′, Desulfomicrobium sp. BL, Desulfomicrobium sp. Bsl6, Desulfomicrobium sp. C4, Desulfomicrobium sp. ‘Clear 59m’, Desulfomicrobium sp. CME2, Desulfomicrobium sp. ‘Delta+’, Desulfomicrobium sp. DsvB, Desulfomicrobium sp. La1.1, Desulfomicrobium sp. MSL65, Desulfomicrobium sp. MSL92, Desulfomicrobium sp. MSL93, Desulfomicrobium sp. MSL94, Desulfomicrobium sp. MSL95, Desulfomicrobium sp. MSL97, Desulfomicrobium sp. MSL98, Desulfomicrobium sp. oral clone BPI-74, Desulfomicrobium sp. P004A, Desulfomicrobium sp. SA2, Desulfomicrobium sp. ‘Scale 10m’, Desulfomicrobium sp. ‘Scale 7m’, Desulfomicrobium sp. ‘Scale 9m’, Desulfomicrobium sp. ‘Scale sediment’, Desulfomicrobium sp. STP10, Desulfomicrobium sp. STP16, Desulfomicrobium thermophilum, Desulfomonas oviles, Desulfonatronovibrio hydrogenovorans, Desulfonatronum cooperativum, Desulfonatronum lacustre, Desulfonatronum sp. DsvA, Desulfonatronum thiodismutans, Desulfonauticus submarinus, Desulfothermus naphthae, Desulfothermus okinawensis, Desulfovermiculus halophilus, Desulfovibrio acrylicus, Desulfovibrio aerotolerans, Desulfovibrio aespoeensis, Desulfovibrio africanus, Desulfovibrio alaskensis, Desulfovibrio alcoholovorans, Desulfovibrio alkalitolerans, Desulfovibrio aminophilus, Desulfovibrio arcticus, Desulfovibrio baarsii, Desulfovibrio bastinii, Desulfovibrio bizertensis, Desulfovibrio brasiliensis, Desulfovibrio burkinensis, Desulfovibrio caledoniensis, Desulfovibrio capillatus, Desulfovibrio carbinolicus, Desulfovibrio carbinoliphilus, Desulfovibrio cavernae, Desulfovibrio cuneatus, Desulfovibrio dechloracetivorans, Desulfovibrio desulfuricans, Desulfovibrio desulfuricans G20, Desulfovibrio desulfuricans subsp. aestuarii, Desulfovibrio desulfuricans subsp. desulfuricans, Desulfovibrio fairfieldensis, Desulfovibrio ferrireducens, Desulfovibrio ferrophilus, Desulfovibrio frigidus, Desulfovibrio fructosovorans, Desulfovibrio gabonensis, Desulfovibrio giganteus, Desulfovibrio gigas, Desulfovibrio gracilis, Desulfovibrio halophilus, Desulfovibrio hydrothermalis, Desulfovibrio indonesiensis, Desulfovibrio inopinatus, Desulfovibrio intestinalis, Desulfovibrio longreachensis, Desulfovibrio longus, Desulfovibrio magneticus, Desulfovibrio marrakechensis, Desulfovibrio mexicanus, Desulfovibrio multispirans, Desulfovibrio oliviopondense, Desulfovibrio oryzae, Desulfovibrio oxyclinae, Desulfovibrio pangongensis, Desulfovibrio piger, Desulfovibrio piger ATCC 29098, Desulfovibrio profundus, Desulfovibrio putealis, Desulfovibrio salexigens, Desulfovibrio senezii, Desulfovibrio simplex, Desulfovibrio singaporenus, Desulfovibrio sp., Desulfovibrio sp. 160, Desulfovibrio sp. 49MC, Desulfovibrio sp. A1, Desulfovibrio sp. A-1, Desulfovibrio sp. A2, Desulfovibrio sp. A4, Desulfovibrio sp. A45, Desulfovibrio sp. ABHU1SB, Desulfovibrio sp. ABHU1SBfatS, Desulfovibrio sp. ABHU2SB, Desulfovibrio sp. Ac5.2, Desulfovibrio sp. An30H-mm, Desulfovibrio sp. An30N-mm, Desulfovibrio sp. AND1, Desulfovibrio sp. ANP3, Desulfovibrio sp. AR1102/00, Desulfovibrio sp. AR1103, Desulfovibrio sp. AR1103/00, Desulfovibrio sp. AR1104/00, Desulfovibrio sp. AR1201/00, Desulfovibrio sp. AR1202/00, Desulfovibrio sp. AR1203/00, Desulfovibrio sp. AR1205/00, Desulfovibrio sp. AR1206/00, Desulfovibrio sp. AS36, Desulfovibrio sp. BBD-10, Desulfovibrio sp. BBD-11, Desulfovibrio sp. BBD-15, Desulfovibrio sp. BBD-16, Desulfovibrio sp. BBD-19, Desulfovibrio sp. BBD-2, Desulfovibrio sp. BBD-22, Desulfovibrio sp. BBD-6, Desulfovibrio sp. ‘Bendigo A’, Desulfovibrio sp. BG50, Desulfovibrio sp. BG6, Desulfovibrio sp. BL-157, Desulfovibrio sp. Bsl2, Desulfovibrio sp. BST-A, Desulfovibrio sp. BST-B, Desulfovibrio sp. BST-C, Desulfovibrio sp. BSY-A, Desulfovibrio sp. BSY-C, Desulfovibrio sp. C/L2, Desulfovibrio sp. CME3, Desulfovibrio sp. D1, Desulfovibrio sp. D4, Desulfovibrio sp. ds2-2, Desulfovibrio sp. DSM 9953, Desulfovibrio sp. DSM12803, Desulfovibrio sp. DsvC, Desulfovibrio sp. E2, Desulfovibrio sp. EBD, Desulfovibrio sp. EX265, Desulfovibrio sp. FD1, Desulfovibrio sp. FHM107, Desulfovibrio sp. FSPa4-5, Desulfovibrio sp. FSR12A, Desulfovibrio sp. FSR12B, Desulfovibrio sp. FSR14A, Desulfovibrio sp. FSR14B, Desulfovibrio sp. FSR17A, Desulfovibrio sp. FSR17B, Desulfovibrio sp. FSRs, Desulfovibrio sp. G05VIII, Desulfovibrio sp. G05XV, Desulfovibrio sp. G05XVI, Desulfovibrio sp. G05XVII, Desulfovibrio sp. G100IX, Desulfovibrio sp. G100V, Desulfovibrio sp. G100VI, Desulfovibrio sp. G200VIII, Desulfovibrio sp. G50VII, Desulfovibrio sp. G50XIV, Desulfovibrio sp. G5V, Desulfovibrio sp. G5VII, Desulfovibrio sp. GWE2, Desulfovibrio sp. HRS-La4, Desulfovibrio sp. HS2, Desulfovibrio sp. IC1, Desulfovibrio sp. IMP-2, Desulfovibrio sp. IrT-JG1-58, Desulfovibrio sp. IrT-JG1-71, Desulfovibrio sp. JCM 14577, Desulfovibrio sp. JD160, Desulfovibrio sp. JG1, Desulfovibrio sp. JG5, Desulfovibrio sp. L3, Desulfovibrio sp. L7, Desulfovibrio sp. La1.2, Desulfovibrio sp. La1.3, Desulfovibrio sp. La1H2, Desulfovibrio sp. La2, Desulfovibrio sp. LB1, Desulfovibrio sp. LM4105, Desulfovibrio sp. LNB1, Desulfovibrio sp. LNB2, Desulfovibrio sp. LS1101/00, Desulfovibrio sp. LS1104/00, Desulfovibrio sp. LS1415/01, Desulfovibrio sp. LS2001/01, Desulfovibrio sp. LS2003/01, Desulfovibrio sp. LVform6, Desulfovibrio sp. LVS-1, Desulfovibrio sp. LVS-10, Desulfovibrio sp. LVS-13, Desulfovibrio sp. LVS-15, Desulfovibrio sp. LVS-21, Desulfovibrio sp. LVS-26, Desulfovibrio sp. M2, Desulfovibrio sp. Met 82, Desulfovibrio sp. midref-29, Desulfovibrio sp. midref-32, Desulfovibrio sp. midref-38, Desulfovibrio sp. midref-41, Desulfovibrio sp. midref-45, Desulfovibrio sp. Mlhm, Desulfovibrio sp. MS, Desulfovibrio sp. MSL10, Desulfovibrio sp. MSL15, Desulfovibrio sp. MUS1, Desulfovibrio sp. N50XVII, Desulfovibrio sp. N5XI, Desulfovibrio sp. N5XII, Desulfovibrio sp. NA104, Desulfovibrio sp. NA202, Desulfovibrio sp. NA302, Desulfovibrio sp. NA81, Desulfovibrio sp. NB21, Desulfovibrio sp. NB62, Desulfovibrio sp. NC301, Desulfovibrio sp. NUS2, Desulfovibrio sp. oral clone BB161, Desulfovibrio sp. P1B2, Desulfovibrio sp. PA35E4, Desulfovibrio sp. PCP1, Desulfovibrio sp. PL35L1, Desulfovibrio sp. Pr1.2, Desulfovibrio sp. PT-2, Desulfovibrio sp. R2, Desulfovibrio sp. Pf35E1, Desulfovibrio sp. SA1, Desulfovibrio sp. SA-6, Desulfovibrio sp. SB1, Desulfovibrio sp. sponge 85CD, Desulfovibrio sp. SRB D2, Desulfovibrio sp. STL12, Desulfovibrio sp. STL13, Desulfovibrio sp. STL2, Desulfovibrio sp. STL3, Desulfovibrio sp. STL7, Desulfovibrio sp. STP1, Desulfovibrio sp. STP4, Desulfovibrio sp. STP5, Desulfovibrio sp. STP7, Desulfovibrio sp. STP8, Desulfovibrio sp. STP9, Desulfovibrio sp. TBP-1, Desulfovibrio sp. UIV, Desulfovibrio sp. UNSW3caefatS, Desulfovibrio sp. W002, Desulfovibrio sp. X, Desulfovibrio sp. ZIRB-2, Desulfovibrio sp. zt10e, Desulfovibrio sp. zt31, Desulfovibrio sulfodismutans, Desulfovibrio termitidis, Desulfovibrio vietnamensis, Desulfovibrio vulgaris, Desulfovibrio vulgaris str. ‘Miyazaki F’, Desulfovibrio vulgaris subsp. oxamicus, Desulfovibrio vulgaris subsp. oxamicus (strain Monticello), Desulfovibrio vulgaris subsp. vulgaris, Desulfovibrio vulgaris subsp. vulgaris DP4, Desulfovibrio vulgaris subsp. vulgaris str. Hildenborough, Desulfovibrio zosterae, Desulfovibrionaceae bacterium MSL79, Desulfovibrionaceae bacterium MSL80, Desulfovibrionaceae bacterium WN022, Desulfovibrionales bacterium H0407_{—}12.1Lac, Desulfovibrionales bacterium H0407_{—}2.3jLac, Desulfovibrionales bacterium H0407_{—}2.3jLac/Ac, Desulfovibrionales bacterium HAW-EB18, Desulfovibrionales bacterium Spi55, Lawsonia cf. intracellularis, Lawsonia intracellularis, and Lawsonia intracellularis PHE/MN1-00.

One or more of the consortiums may include a microorganism selected from Desulfatibacillum aliphaticivorans, Desulfatibacillum alkenivorans, Desulfatibacillum alkenivorans AK-01, Desulfatibacillum sp. Pnd3, Desulfatibacillus olefinivorans, Desulfoarculus sp. BG74, Desulfobacter curvatus, Desulfobacter halotolerans, Desulfobacter hydrogenophilus, Desulfobacter latus, Desulfobacter postgatei, Desulfobacter psychrotolerans, Desulfobacter sp., Desulfobacter sp. ASv25, Desulfobacter sp. BG23, Desulfobacter sp. BG72, Desulfobacter sp. BG8, Desulfobacter sp. DSM 2035, Desulfobacter sp. DSM 2057, Desulfobacter vibrioformis, Desulfobacteraceae bacterium 166, Desulfobacteraceae bacterium 171, Desulfobacteraceae bacterium 175, Desulfobacteraceae bacterium MSL53, Desulfobacteraceae bacterium MSL71, Desulfobacterium anilini, Desulfobacterium autotrophicum, Desulfobacterium autotrophicum HRM2, Desulfobacterium catecholicum, Desulfobacterium cetonicum, Desulfobacterium corrodens, Desulfobacterium indolicum, Desulfobacterium sp. AK1, Desulfobacterium sp. BG18, Desulfobacterium sp. BG33, Desulfobacterium sp. BSv41, Desulfobacterium sp. LSv25, Desulfobacterium sp. MB-2005, Desulfobacterium sp. PM4, Desulfobacterium vacuolatum, Desulfobacterium zeppelinii, Desulfobacula phenolica, Desulfobacula toluolica, Desulfobotulus sapovorans, Desulfobotulus sp. BG14, Desulfobulbus elongates, Desulfobulbus japonicus, Desulfobulbus marinus, Desulfobulbus mediterraneus, Desulfobulbus propionicus, Desulfobulbus rhabdoformis, Desulfobulbus sp. ‘Ace 16m’, Desulfobulbus sp. BG25, Desulfobulbus sp. ‘Clear 54m’, Desulfobulbus sp. DSM 2033, Desulfobulbus sp. DSM 2058, Desulfobulbus sp. LB2, Desulfobulbus sp. ‘McCal 25m’, Desulfobulbus sp. oral clone CH031, Desulfobulbus sp. oral clone R004, Desulfobulbus sp. RPf35L17, Desulfocapsa sp. Cad626, Desulfocapsa sp. La4.1, Desulfocapsa sulfexigens, Desulfocapsa thiozymogenes, Desulfocella halophila, Desulfocella sp. DSM 2056, Desulfococcus biacutus, Desulfococcus multivorans, Desulfococcus niacini, Desulfococcus oleovorans, Desulfococcus oleovorans Hxd3, Desulfococcus sp. DSM 8541, Desulfofaba fastidiosa, Desulfofaba gelida, Desulfofaba hansenii, Desulfofrigus fragile, Desulfofrigus oceanense, Desulfofrigus sp. HRS-La3x, Desulfofrigus sp. J152, Desulfofrigus sp. NA201, Desulfofrigus sp. NB81, Desulfofustis glycolicus, Desulfonema ishimotonii, Desulfonema limicola, Desulfonema magnum, Desulfopila aestuarii, Desulforegula conservatrix, Desulforhopalus singaporensis, Desulforhopalus sp. LSv20, Desulforhopalus vacuolatus, Desulfosalina propionicus, Desulfosarcina sp. CME1, Desulfosarcina variabilis, Desulfospira joergensenii, Desulfotalea arctica, Desulfotalea psychrophila, Desulfotalea psychrophila LSv54, Desulfotalea sp. NA22, Desulfotalea sp. SFA4, Desulfotignum balticum, Desulfotignum phosphitoxidans, Desulfotignum sp. DSM 7120, and Desulfotignum toluolica.

The consortiums may include microorganism from the family Clostridia, at least some of which may participate in the conversion of complex hydrocarbon substrates to acetate groups, hydrogen gas, and carbon dioxide. Two genera of Clostridia bacteria, Acetobacterium and Fusibacter, placed in hydrocarbon-bearing environments (e.g., underground oil storage cavities, oil producing wells, etc.) may participate in the conversion of in-situ complex hydrocarbon substrates (e.g., oil) to acetate, methane and/or hydrogen gas. The full taxonomic identification of each genera are as follows:

| Domain | Phylum | Class | Order | Family | Genus |
|---|---|---|---|---|---|
| Bacteria | Firmicutes | Clostridia | Clostridiales | Eubacteriaceae | Acetobacterium |
| Bacteria | Firmicutes | Clostridia | Clostridiales | Peptostreptococcaceae | Fusibacter |

Both genera may include species that produce acetate through the fermentation of starting hydrocarbons. Acetobacterium species may also generate acetate through homoacetogenesis using hydrogen gas and carbon dioxide. Both genera may also include species that generate hydrogen gas, with some Acetobacterium species having a syntrophic relationship with methanogens when producing the hydrogen.

These Clostridia consortiums may include one or more genera of syntrophomonadaceae microorganisms such as Aminiphilus circumscriptus, Aminobacterium colombiense, Aminobacterium mobile, Aminomonas paucivorans, Anaerobaculum mobile, Anaerobaculum sp. TERI 001, Anaerobaculum thermoterrnum, Anaerobranca californiensis, Anaerobranca gottschalkii, Anaerobranca horikoshii, Anaerobranca zavarzinii, Caldicellulosiruptor acetigenus, Caldicellulosiruptor hydrothermalis, Caldicellulosiruptor kristjanssonii, Caldicellulosiruptor kronotskiensis, Caldicellulosiruptor lactoaceticus, Caldicellulosiruptor owensensis, Caldicellulosiruptor saccharolyticus, Caldicellulosiruptor saccharolyticus DSM 8903, Caldicellulosiruptor sp. Rt69B.1, Caldicellulosiruptor sp. Rt8B.4, Caldicellulosiruptor sp. Tok7B.1, Caldicellulosiruptor sp. YI5, Candidatus Contubernalis alkalaceticum, Carboxydocella ferrireducens, Carboxydocella sp. 1244, Carboxydocella sp. 1503, Carboxydocella sp. 930, Carboxydocella sp. 961, Carboxydocella sporoproducens, Carboxydocella thermautotrophica, Dethiosulfovibrio acidaminovorans, Dethiosulfovibrio marinus, Dethiosulfovibrio peptidovorans, Dethiosulfovibrio peptidovorans DSM 11002, Dethiosulfovibrio russensis, Pelospora glutarica, Syntrophomonadaceae bacterium CDA4, Syntrophomonadaceae genomosp. P1, Syntrophomonas cellicola, Syntrophomonas curvata, Syntrophomonas erecta, Syntrophomonas erecta subsp. sporosyntropha, Syntrophomonas palmitatica, Syntrophomonas sapovorans, Syntrophomonas sp. MGB-C1, Syntrophomonas sp. TB-6, Syntrophomonas sporosyntrophas, Syntrophomonas wolfei, Syntrophomonas wolfei subsp. methylbutyratica, Syntrophomonas wolfei subsp. saponavida, Syntrophomonas wolfei subsp. wolfei, Syntrophomonas wolfei subsp. wolfei str. Goettingen, Syntrophomonas zehnderi, Syntrophospora bryantii, Syntrophothermus lipocalidus, Thermaerobacter litoralis, Thermaerobacter marianensis, Thermaerobacter nagasakiensis, Thermaerobacter sp. C4-1, Thermaerobacter sp. enrichment clone A20, Thermaerobacter sp. enrichment clone A7, Thermaerobacter subterraneus, Thermanaerovibrio acidaminovorans, Thermanaerovibrio velox, Thermosyntropha lipolytica, and/or Thermovirga lienii.

These Clostridia consortiums may include one or more genera of acetobacterium microorganisms such as Acetobacterium bakii, Acetobacterium carbinolicum, Acetobacterium carbinolicum subsp. kysingense, Acetobacterium dehalogenans, Acetobacterium fimetarium, Acetobacterium malicum, Acetobacterium paludosum, Acetobacterium psammolithicum, Acetobacterium sp. Ha4, Acetobacterium sp. HAAP-1, Acetobacterium sp. LS1, Acetobacterium sp. LS2, Acetobacterium sp. Schreyahn_Kolonie_Aster_{—}3.2, Acetobacterium sp. TM20-2, Acetobacterium submarinus, Acetobacterium tundrae, Acetobacterium wieringae, Acetobacterium woodii, Alkalibacter saccharofermentans, Alkalibacter sp. TC3, Anaerofustis stercorihominis, Anaerofustis stercorihominis DSM 17244, Anaerovorax odorimutans, Eubacteriaceae bacterium BL-152, Eubacteriaceae bacterium P4P_{—}50 P4, Eubacteriaceae bacterium WK012, Eubacteriaceae bacterium WN037, Eubacteriaceae oral clone MCE10_{—}174, Eubacteriaceae oral clone P2PB_{—}46 P3, Eubacteriaceae oral clone P2PC_{—}29 P2, Eubacterium acidaminophilum, Eubacterium aggregans, Eubacterium albensis, Eubacterium angustum, Eubacterium barkeri, Eubacterium brachy, Eubacterium budayi, Eubacterium callanderi, Eubacterium cellulosolvens, Eubacterium cf. saburreum oral strain C27KA, Eubacterium combesii, Eubacterium contortum, Eubacterium coprostanoligenes, Eubacterium desmolans, Eubacterium eligens, Eubacterium fissicatena, Eubacterium hallii, Eubacterium hallii DSM 3353, Eubacterium infirmum, Eubacterium limosum, Eubacterium minutum, Eubacterium moniliforme, Eubacterium multiforme, Eubacterium nitritogenes, Eubacterium nodatum, Eubacterium oxidoreducens, Eubacterium pectinii, Eubacterium plautii, Eubacterium plautii ATCC 29863, Eubacterium plexicaudatum, Eubacterium pyruvativorans, Eubacterium ramulus, Eubacterium rectale, Eubacterium ruminantium, Eubacterium saburreum-like sp. oral clone CK004, Eubacterium saphenum, Eubacterium siraeum, Eubacterium siraeum DSM 15702, Eubacterium sp., Eubacterium sp. 1275b, Eubacterium sp. 4c, Eubacterium sp. A-44, Eubacterium sp. ADS17, Eubacterium sp. ARC-2, Eubacterium sp. BBDP17, Eubacterium sp. BBDP67, Eubacterium sp. BBDP70, Eubacterium sp. BL13, Eubacterium sp. BL22, Eubacterium sp. BL38, Eubacterium sp. C124b, Eubacterium sp. C12b, Eubacterium sp. C2, Eubacterium sp. CB4, Eubacterium sp. CJ70, Eubacterium sp. cL-10-1-3, Eubacterium sp. cp03.14, Eubacterium sp. Cs1 Van, Eubacterium sp. cTPY-18, Eubacterium sp. F1, Eubacterium sp. KE2-08, Eubacterium sp. L2-7, Eubacterium sp. oral clone 3RH-1, Eubacterium sp. oral clone BB142, Eubacterium sp. oral clone BE088, Eubacterium sp. oral clone BP1-11, Eubacterium sp. oral clone BP1-2, Eubacterium sp. oral clone BP1-20, Eubacterium sp. oral clone BP1-24, Eubacterium sp. oral clone BP1-26, Eubacterium sp. oral clone BP1-27, Eubacterium sp. oral clone BP1-3, Eubacterium sp. oral clone BP1-31, Eubacterium sp. oral clone BP1-32, Eubacterium sp. oral clone BP1-34, Eubacterium sp. oral clone BP1-41, Eubacterium sp. oral clone BP1-47, Eubacterium sp. oral clone BP1-57, Eubacterium sp. oral clone BP1-61, Eubacterium sp. oral clone BP1-62, Eubacterium sp. oral clone BP1-69, Eubacterium sp. oral clone BP1-75, Eubacterium sp. oral clone BP1-77, Eubacterium sp. oral clone BP1-82, Eubacterium sp. oral clone BP1-89, Eubacterium sp. oral clone BP1-93, Eubacterium sp. oral clone BP2-88, Eubacterium sp. oral clone BR088, Eubacterium sp. oral clone B5091, Eubacterium sp. oral clone BU014, Eubacterium sp. oral clone BU061, Eubacterium sp. oral clone CK047, Eubacterium sp. oral clone DA014, Eubacterium sp. oral clone DN050, Eubacterium sp. oral clone D0008, Eubacterium sp. oral clone DO016, Eubacterium sp. oral clone DZ073, Eubacterium sp. oral clone EH006, Eubacterium sp. oral clone EI074, Eubacterium sp. oral clone EW049, Eubacterium sp. oral clone EW053, Eubacterium sp. oral clone FX028, Eubacterium sp. oral clone FX033, Eubacterium sp. oral clone GI038, Eubacterium sp. oral clone HU029, Eubacterium sp. oral clone IR009, Eubacterium sp. oral clone JH012, Eubacterium sp. oral clone JI012, Eubacterium sp. oral clone JN088, Eubacterium sp. oral clone JS001, Eubacterium sp. oral clone OH3A, Eubacterium sp. oral strain A03MT, Eubacterium sp. oral strain A35MT, Eubacterium sp. Pei061, Eubacterium sp. PG-04, Eubacterium sp. SG121, Eubacterium sp. SG1213, Eubacterium sp. SG1215, Eubacterium sp. SG122, Eubacterium sp. SG123, Eubacterium sp. TW2, Eubacterium sulci, Eubacterium tarantellae, Eubacterium tenue, Eubacterium thermomarinus, Eubacterium uniforme, Eubacterium ventriosum, Eubacterium ventriosum ATCC 27560, Eubacterium xylanophilum, Eubacterium yurii, Eubacterium yurii subsp. margaretiae, Eubacterium yurii subsp. schtitka, Eubacterium yurii subsp. yurii, Mogibacterium diversum, Mogibacterium neglectum, Mogibacterium pumilum, Mogibacterium sp. oral clone BP1-36, Mogibacterium sp. oral clone BP1-95, Mogibacterium sp. oral clone BP1-96, Mogibacterium timidum, Mogibacterium vescum, Pseudoramibacter alactolyticus, and/or Pseudoramibacter sp. oral clone BP1-8.

These Clostridia consortiums may include one or more genera of fusibacter microorganisms such as Acetanaerobacter sp. Iso-W4, Anaerococcus hydrogenalis, Anaerococcus lactolyticus, Anaerococcus murdochii, Anaerococcus octavius, Anaerococcus prevotii, Anaerococcus sp. BG1, Anaerococcus sp. BG2, Anaerococcus sp. gpac028, Anaerococcus sp. gpac047, Anaerococcus sp. gpac104, Anaerococcus sp. gpac126, Anaerococcus sp. gpac137, Anaerococcus sp. gpac155, Anaerococcus sp. gpac165, Anaerococcus sp. gpac199, Anaerococcus sp. gpac215, Anaerococcus tetradius, and Anaerococcus vaginalis, Filifactor alocis, Filifactor sp. oral clone BP1-37, Filifactor sp. oral clone BP1-51, Filifactor sp. oral clone BP1-54, Filifactor sp. oral clone BP1-58, Filifactor sp. oral clone BP1-67, Filifactor sp. oral clone BP1-81, Filifactor sp. oral clone BP1-88, Filifactor villosus, Finegoldia magna, Finegoldia magna ATCC 29328, Fusibacter paucivorans, Fusibacter sp. SA1, Gallicola barnesae, Helcococcus kunzii, Helcococcus ovis, Helcococcus pyogenes, Helcococcus sp. DRBC0899CHER3, Helcococcus sueciensis, Peptoniphilus asaccharolyticus, Peptoniphilus gorbachii, Peptoniphilus harei, Peptoniphilus indolicus, Peptoniphilus ivorii, Peptoniphilus lacrimalis, Peptoniphilus olsenii, Peptoniphilus sp. 2002-2300004, Peptoniphilus sp. 2002-38328, Peptoniphilus sp. BG3, Peptoniphilus sp. BG4, Peptoniphilus sp. BGS, Peptoniphilus sp. gpac003, Peptoniphilus sp. gpac007, Peptoniphilus sp. gpac018A, Peptoniphilus sp. gpac018B, Peptoniphilus sp. gpac055, Peptoniphilus sp. gpac063, Peptoniphilus sp. gpac077, Peptoniphilus sp. gpac121, Peptoniphilus sp. gpac148, Peptostreptococcaceae bacterium 19gly3, Peptostreptococcaceae bacterium STV110602, Peptostreptococcaceae bacterium WN036, Peptostreptococcaceae bacterium WN082, Peptostreptococcus anaerobius, Peptostreptococcus genosp. 4, Peptostreptococcus micros, Peptostreptococcus micros ATCC 33270, Peptostreptococcus sp., Peptostreptococcus sp. 1018, Peptostreptococcus sp. 2002-69396, Peptostreptococcus sp. C27, Peptostreptococcus sp. CCUG 42997, Peptostreptococcus sp. cp10.23, Peptostreptococcus sp. D1, Peptostreptococcus sp. E3_{—}32, Peptostreptococcus sp. MDA2346-2, Peptostreptococcus sp. oral clone AJ062, Peptostreptococcus sp. oral clone AP24, Peptostreptococcus sp. oral clone BP1-1, Peptostreptococcus sp. oral clone BP1-18, Peptostreptococcus sp. oral clone BP1-22, Peptostreptococcus sp. oral clone BP1-50, Peptostreptococcus sp. oral clone BP1-59, Peptostreptococcus sp. oral clone BP1-72, Peptostreptococcus sp. oral clone BP1-73, Peptostreptococcus sp. oral clone BP1-84, Peptostreptococcus sp. oral clone BS044, Peptostreptococcus sp. oral clone CK035, Peptostreptococcus sp. oral clone EX153, Peptostreptococcus sp. oral clone FG014, Peptostreptococcus sp. oral clone FJ023, Peptostreptococcus sp. oral clone FL008, Peptostreptococcus sp. oral clone HE064, Peptostreptococcus sp. oral clone P4PA_{—}156 P4, Peptostreptococcus sp. P4P_{—}31 P3, Peptostreptococcus sp. SI, Peptostreptococcus stomatis, Sedimentibacter hongkongensis, Sedimentibacter hydroxybenzoicus, Sedimentibacter saalensis, Sedimentibacter sp. B4, Sedimentibacter sp. BAF1, Sedimentibacter sp. BD7-4, Sedimentibacter sp. BRS2, Sedimentibacter sp. C7, Sedimentibacter sp. D7, Sedimentibacter sp. enrichment clone 2Ben5, Sedimentibacter sp. enrichment clone Lace6, Sedimentibacter sp. enrichment clone Lace8, Sedimentibacter sp. IMPC3, Sedimentibacter sp. JN18_A14_H, Sedimentibacter sp. JN18_V27_I, Sporanaerobacter acetigenes, Tissierella creatinini, Tissierella creatinophila, Tissierella praeacuta, and/or Tissierella sp.

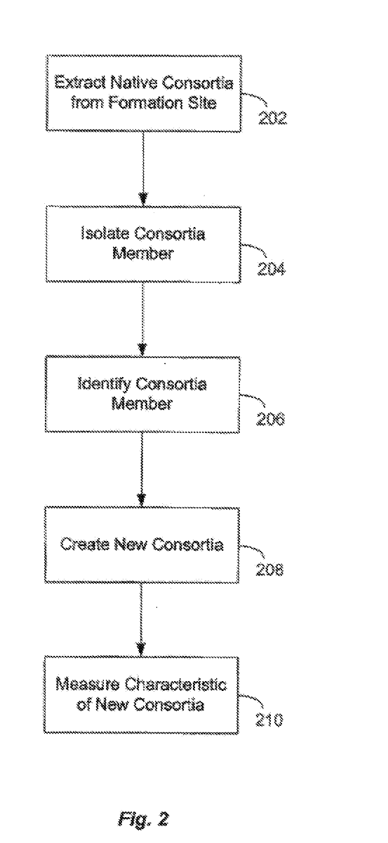

FIG. 2 shows a flowchart with method steps for making and measuring the characteristics of a consortia. In the embodiment shown, the method starts with extracting native consortia from a formation site 202. The consortia may be taken from solid substrate at the site and/or formation water stored in the site. Subsets and/or individual members may be isolated from the extracted consortia 204. Isolation techniques may include any known in the art as well as those described in U.S. Patent Application entitled “Systems and methods for the isolation of microorganisms in hydrocarbon deposits” by Gary Vanzin filed on the same day as the instant application, the entire contents of which are hereby incorporated by this reference for all purposes. The consortia members may also be identified 206, either before or after they are isolated. Identification techniques may include identification of signature proteins, and/or nucleic acid sequences that identify the presence of the microorganism.

The method may also include combining members and/or subsets of the native consortia to form a new consortia 208. Genetically modified microorganisms not found in any native consortia may also be introduced. Characteristic of the new consortia may be measured 210, such as the consumption rate of carbonaceous material and/or the production rate of metabolite (e.g., methane). Measured characteristics may also involve the response of the new consortia to amendments made to the consortia's environments, such as changes in temperature, pH, oxidation potential (Eh), nutrient concentrations, salinity, metal ion concentrations, etc.

From the information gleaned from these and other studies, consortia may be produced with enhanced rates of metabolic activity for in situ conversions of carbonaceous materials in sub-surface formations to hydrocarbons with higher mol. % hydrogen. These consortia may be formed by isolating and combining individual consortia (i.e., subsets of the consortia) or even individual microbial species. They may also be formed by amending one or more conditions in the consortia environment that favor the growth of one species or consortium over another. These amendments may include the introduction of a growth inhibitor that slows or stops the growth of one or more microbial species, and the introduction of a growth stimulant that increases the growth rate of one or more microbial species.

### Experimental

Measurements were conducted to compare the percentage of Desulfuromonas, Fusibacter, and Acetobacterium in a microorganism consortium with the methanogenesis rate for that consortium. The % Desulfuromonas % Fusibacter, % Acetobacterium and methanogenesis rate were measured for 12 microorganism consortiums. Table I below provides additional information about the consortiums, their methanogenesis rates, and the % Desulfuromonas, % Fusibacter, and % Acetobacterium in the consortiums:

TABLE I
Comparison of Microorganism Population Percentages with Methanogenesis Rate

| | | Methanogenesis | % cosortium | % cosortium | % cosortium |
|---|---|---|---|---|---|
| Consortium | Source | rate (mmoles | comprised of | comprised of | comprised of |
| name | material | CH₄/g coal/day) | Desulfuromonas | Fusibacter | Acetobacterium |
| TRP64ME001 | 41M-2083 coal | 1.24 | 2.70 | 2.70 | 64.86 |
| | and water | | | | |
| TRP64ME002 | 23C-2483 coal | 1.37 | 4.76 | 2.38 | 76.19 |
| | and water | | | | |
| TRP64ME006 | 41M-2083 coal | 3.59 | 33.33 | 12.82 | 46.15 |
| | 23M-2283 water | | | | |
| TRP64ME007 | 23M-2283 coal | 3.096 | 29.73 | 27.03 | 37.84 |
| | and water | | | | |
| TRP64ME008 | 23C-2483 coal | 3.929 | 16.67 | 16.67 | 50.00 |
| | and water | | | | |
| TRP64ME009 | 23C-2483 coal | 1.831 | 9.52 | 9.52 | 71.43 |
| | and water | | | | |
| TRP64ME010 | 23M-2283 coal | 4.301 | 14.66 | 17.07 | 60.98 |
| | and water | | | | |
| TRP64ME012 | 32D1-2183 coal | 1.212 | 11.54 | 3.85 | 67.31 |
| | and water | | | | |
| TRP64ME013 | 32D1-2183 coal | 1.276 | 13.89 | 5.56 | 58.33 |
| | and water | | | | |
| TRP64ME014 | 41M-2083 coal | 1.202 | 6.98 | 9.30 | 65.12 |
| | and water | | | | |
| TRP64ME015 | 41M-2083 Coal | 4.09 | 53.33 | 13.33 | 20.00 |
| | 23M-2283 Water | | | | |
| TRP64ME016 | 41M-2083 Coal | 3.803 | 18.18 | 13.64 | 59.09 |
| | 23M-2283 Water | | | | |

### Desulfuromonas Measurements and Results

The % Desulfuromonas was measured by sequencing 16s rDNA found in each consortium. 16s rDNA allows the Desulfuromonas to be distinguished from other microorganisms in the consortium and quantified as a percentage of the total population of the microorganisms in the consortium. One uncertainty involved with this measurement technique is that 16s rDNA sequence is practically indistinguishable between Desulfuromonas and another microorganism genus called Pelobacter. However, Desulfuromonas is considered the more universal genera in both the lab and the field, and Desulfuromonas is more likely to be found where carbonaceous material is being digested through hydrocarbon metabolism. For both these reasons, it is believed that the 16s rDNA measurements performed here mostly (if not exclusively) represent Desulfuromonas.

The rate of methanogenesis for each of the consortiums was measured by placing the consortium in slurry bottles and measuring the methane concentration in the headspace above the liquid as a function of time. Sealed anaerobic cultures were established in 13 ml vessels containing 0.6 grams of sterile anaerobic Tongue River coal and 2.5 ml of sterile anaerobic Tongue River formation water. These sterile media bottles were inoculated with approximately 1×10⁴ cells from currently growing cultures comprised of coal and water as noted in Table 1 as “source material”. Data used for methanogenesis rates were the maximum rate obtained between days 21 and 33 of culture growth. Cultures were monitored for methanogenesis for 93 days.

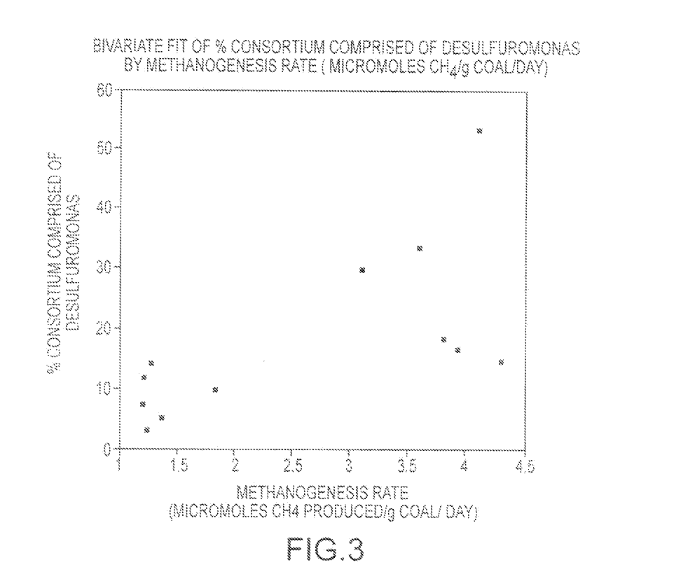

FIG. 3 is a plot of the methanogensis rate (μmols of methane/gram of coal/day) as a function of the percentage of Desulfuromonas in a microorganism consortium. The plot shows an increased % Desulfuromonas correlates with statistically higher methanogenesis rates for the consortium. This was confirmed by a statistical analysis of the plot, which had a Student's T-test p-value of 0.0178 (<0.05 is statistically significant). Statistical analysis was performed using JMP Statistical Discovery™ software.

### Fusibacter Measurements and Results

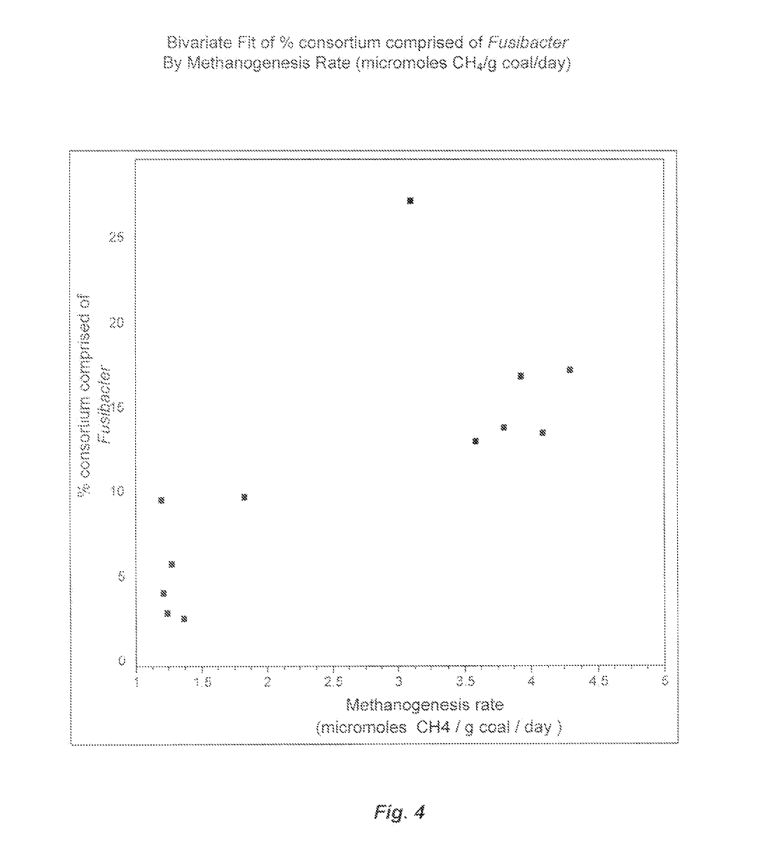

The identification and concentration measurements for the Fusibacter, as well as the measurements of the methanogenesis rate, were the same as used for the Desulfuromonas. FIG. 4 shows the methanogensis rate (μmols of methane/gram of coal/day) as a function of the percentage of Fusibacter to have a similar correlation as Desulfuromonas in FIG. 3. This was confirmed by a statistical analysis of the plot, which had a Student's T-test p-value of 0.0064 (<0.05 is statistically significant). Statistical analysis was performed using JMP Statistical Discovery™ software. Thus, like Desulfuromonas, an increased % Fusibacter in a microorganism consortium correlates with statistically higher methanogenesis rates.

### Acetobacterium Measurements and Results

The identification and concentration measurements for the Acetobacterium, as well as the measurements of the methanogenesis rate, were the same as used for the Desulfuromonas. As the data shows in Table 1, Acetobacterium is a large and important component of the highly methanogenic coal metabolizing consortiums. For the 12 consortiums listed in Table 1, the dominant genus identified was Acetobacterium, which averaged 56% of the microorganisms in the consortium:

| | Methanogenesis Rate | % |
|---|---|---|
| Value | (micromoles CH₄/g coal/day) | Acetobacterium |
| Mean | 2.5783333 | 56.441667 |
| Std. Deviation | 1.3189258 | 15.758276 |
| Std. Error Mean | 0.3807411 | 4.5490223 |
| Upper 95% Mean | 3.4163388 | 66.453997 |
| Lower 95% Mean | 1.7403278 | 46.429336 |
| Population Size (N) | 12 | 12 |

The size of the Acetobacterium populations in the most methanogenically active consortiums at least shows a positive correlation between Acetobacterium and methanogenesis rate. Thus, Acetobacterium may be included in one or more consortiums identified here for enhancing the methanogenesis rate in a formation site.

Having described several embodiments, it will be recognized by those of skill in the art that various modifications, alternative constructions, and equivalents may be used without departing from the spirit of the invention. Additionally, a number of well known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the electrode” includes reference to one or more electrodes and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. An isolated microbial consortia comprising:
a first-bite microbial consortium that converts a starting hydrocarbon comprising a complex hydrocarbon into two or more first-bite metabolic products; and
a downstream microbial consortium that converts a starting hydrocarbon metabolic product into a downstream metabolic product, wherein the downstream metabolic product has a greater mol. % hydrogen than the starting hydrocarbon, and wherein the first-bite microbial consortium or the downstream microbial consortium comprises one or more species of Desulfuromonas.

2. The isolated microbial consortia of claim 1, wherein both the first-bite microbial consortium and the downstream microbial consortium are anaerobic microbial consortiums.

3. The isolated microbial consortia of claim 1, wherein at least one of the first-bite metabolic products is the starting hydrocarbon metabolic product.

4. The isolated microbial consortia of claim 1, wherein the consortia further comprises an intermediate microbial consortium that converts the first-bite metabolic product into the starting hydrocarbon metabolic product.

5. The isolated microbial consortia of claim 4, wherein the intermediate microbial consortium comprises a plurality of microorganism species that converts the first-bite metabolic product into the starting hydrocarbon metabolic product through a plurality of intermediate metabolic products.

6. The isolated microbial consortia of claim 5, wherein at least one of the plurality of microorganism species is a species of Desulfuromonas.

7. The isolated microbial consortia of claim 1, wherein the starting hydrocarbon comprises coal, oil, kerogen, peat, lignite, oil shale, tar sands, bitumen, or tar.

8. The isolated microbial consortia of claim 1, wherein the downstream metabolic product comprises an organic acid, an alcohol, an amine, a straight or branched hydrocarbon, or an aromatic hydrocarbon.

9. The isolated microbial consortia of claim 1, wherein the downstream metabolic product comprises methane.

10. The isolated microbial consortia of claim 1, wherein the starting hydrocarbon metabolic product comprises a formate compound, an acetate compound, a benzoate compound, an alcohol, or an organic acid.

11. The isolated microbial consortia of claim 5, wherein the intermediate metabolic products comprise CO or CO₂.

12. An isolated microbial consortia for biogenic methane production, the consortia comprising:
a first microbial consortium that converts a starting hydrocarbon into one or more intermediate compounds;
a second microbial consortium that converts at least one type of the intermediate compounds into CO₂ and H₂; and
a third microbial consortium that converts the CO₂ and H₂ into methane and water,
wherein at least one of the first, second and third microbial consortiums comprises at least one species of Desulfuromonas.

13. The microorganism consortia of claim 12, wherein the starting hydrocarbon comprises coal, oil, kerogen, peat, lignite, oil shale, tar sands, bitumen, or tar, and wherein the intermediate compound comprises a formate compound, an acetate compound, a benzoate compound, an alcohol, or an organic acid.

14. The microorganism consortia of claim 12, wherein the first microbial consortium comprises a species of Desulfuromonas.

15. The microorganism consortia of claim 12, wherein the second microbial consortium comprises a species of Desulfuromonas.

16. The microorganism consortia of claim 12, wherein the third microbial consortium comprises a species of Desulfuromonas.

17. The microorganism consortia of claim 12, wherein the first, second and third consortiums consist of anaerobic microorganisms.

18. An isolated microbial consortia for biogenic methane production, the consortia comprising:
a first microbial consortium that converts a starting hydrocarbon into one or more intermediate compounds;
a second microbial consortium that converts at least one type of the intermediate compounds into acetate; and
a third microbial consortium that converts the acetate into methane and water,
wherein at least one of the first, second and third microbial consortiums comprises at least one species of Desulfuromonas.

19. The microbial consortia of claim 18, wherein the third microbial consortium comprises a first group of microorganisms that convert acetate into carbon dioxide and free hydrogen, and a second group of microorganisms that convert the carbon dioxide and free hydrogen into methane and water.

20. The microbial consortia of claim 19, wherein the third microbial consortia comprises a species of Desulfuromonas.

21. The microorganism consortia of claim 18, wherein the first, second and third consortiums consist of anaerobic microorganisms.

22. An isolated microbial consortia comprising:
a first-bite microbial consortium that converts a starting hydrocarbon comprising a complex hydrocarbon into two or more first-bite metabolic products; and
a downstream microbial consortium that converts a starting hydrocarbon metabolic product into a downstream metabolic product, wherein the downstream metabolic product has a greater mol. % hydrogen than the starting hydrocarbon, and wherein the first-bite microbial consortium or the downstream microbial consortium comprises one or more species of Fusibacter.

23. An isolated microbial consortia comprising:
a first-bite microbial consortium that converts a starting hydrocarbon comprising a complex hydrocarbon into two or more first-bite metabolic products; and
a downstream microbial consortium that converts a starting hydrocarbon metabolic product into a downstream metabolic product, wherein the downstream metabolic product has a greater mol. % hydrogen than the starting hydrocarbon, and wherein the first-bite microbial consortium or the downstream microbial consortium comprises one or more species of Acetobacterium.
